(19) United States
(12) Patent Application Publication
(10) Pub. No.: US 2025/0143915 A1
(43) Pub. Date: May 8, 2025

(54) FLUID COLLECTION ASSEMBLIES INCLUDING A CONDUIT HAVING A SHAPE MEMORY MATERIAL

(71) Applicant: PUREWICK CORPORATION, Covington, GA (US)

(72) Inventors: Zhihui Yin, Lilburn, GA (US); Steven Michael Altomare, Marietta, GA (US)

(21) Appl. No.: 18/931,853

(22) Filed: Oct. 30, 2024

Related U.S. Application Data
(60) Provisional application No. 63/596,012, filed on Nov. 3, 2023.

(51) Int. Cl. A61F 5/451; A61F 5/44

(52) U.S. Cl. CPC A61F 5/451; A61F 5/4404

(57) ABSTRACT

An example fluid collection assembly includes a fluid impermeable layer that at least defines a chamber, at least one opening, and a fluid outlet. The fluid collection assembly includes at least one porous material disposed in the chamber and a multiple lumen conduit disposed in the at least one porous material. The multiple lumen conduit includes fluid lumen and a shape memory lumen having a shape memory material retained therein by a stop plug and an end plug.

### CROSS REFERENCE TO RELATED APPLICATIONS

This application claims priority to U.S. Provisional Patent Application No. 63/596,012 filed on 3 Nov. 2023, the disclosure of which is incorporated herein in its entirety by this reference.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

Embodiments are directed to fluid collection assemblies including a multiple lumen conduit having a shape memory material disposed in the shape memory lumen therein, fluid collection systems including the same, and methods of using and forming the same. In an embodiment, a fluid collection assembly is disclosed. The fluid collection assembly includes a fluid impermeable layer defining a chamber therein, at least one opening therethrough, and a fluid outlet. The fluid collection assembly includes at least one porous material disposed in the chamber. The fluid collection assembly includes a conduit disposed in the at least one porous material, the conduit including a multiple lumen configuration having a fluid lumen and a shape memory lumen. The fluid collection assembly includes a shape memory material disposed in the shape memory lumen. The fluid collection assembly includes a stop plug and end plug disposed in the shape memory lumen, wherein the stop plug and end plug are disposed at opposite ends of the shape memory material in the shape memory lumen to prevent longitudinal movement of the shape memory material within the shape memory lumen.

In an embodiment, a fluid collection system is disclosed. The fluid collection system includes a fluid collection assembly. The fluid collection assembly includes a fluid impermeable layer defining a chamber therein, at least one opening therethrough, and a fluid outlet. The fluid collection assembly includes at least one porous material disposed in the chamber. The fluid collection assembly includes a conduit disposed in the at least one porous material, the conduit including a multiple lumen configuration having a fluid lumen and a shape memory lumen. The fluid collection assembly includes a shape memory material disposed in the shape memory lumen. The fluid collection assembly includes a stop plug and end plug disposed in the shape memory lumen, wherein the stop plug and end plug are disposed at opposite ends of the shape memory material in the shape memory lumen to prevent longitudinal movement of the shape memory material within the shape memory lumen. The fluid collection system also includes a fluid storage container and a vacuum source. The chamber of the fluid collection assembly, the fluid storage container, and the vacuum source are in fluid communication with each other such that when one or more bodily fluids are present in the chamber, a suction provided from the vacuum source to the chamber of the fluid collection assembly removes the one or more bodily fluids from the chamber and deposits the bodily fluids in the fluid storage container.

In an embodiment, a method of collecting bodily fluid using a fluid collection assembly is disclosed. The method includes forming the fluid collection assembly into a selected shape by bending the shape memory material in the multiple lumen conduit thereof. The method includes positioning the opening of the fluid collection assembly adjacent to or on the urethra of a wearer. The method includes receiving bodily fluid of the wearer in the fluid collection assembly. The method also includes receiving one or more bodily fluids from the urethral opening through the at least one opening and into the at least one porous material.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIGS. 1B and 1C are cross-sectional schematics of the fluid collection assembly taken along planes 1B-1B and IC-1C, respectively, shown in FIG. 1A

FIG. 1D is a cross-sectional schematic of a portion of the fluid collection assembly taken from the box illustrated in FIG. 1C, according to an embodiment.

FIG. 1E is a cross-sectional view of the conduit, according to an embodiment.

FIG. 2A is a cross-sectional schematic of a fluid collection assembly, according to an embodiment.

FIG. 2B is a cross-sectional schematic of the fluid collection assembly of FIG. 2A taken along plane 2B-2B.

FIG. 3 is a cross-sectional schematic of a fluid collection assembly, according to an embodiment.

FIG. 4 is a block diagram of a fluid collection system for fluid collection, according to an embodiment.

### DETAILED DESCRIPTION

Embodiments are directed to fluid collection assemblies including a multiple lumen conduit having a shape memory material disposed in at least one of the lumen, fluid collection systems including the same, and methods of using and forming the same. An example fluid collection assembly includes a fluid impermeable layer (e.g., a fluid impermeable barrier) that at least defines a chamber, at least one opening, and a fluid outlet. The fluid collection assembly also includes at least one porous material disposed in the chamber. The fluid collection assembly also includes a multiple lumen conduit having a shape memory material disposed in at least one of the lumen where the shape memory material is contained therein by an end plug and a stop plug in the lumen.

During use, the fluid collection assembly may be positioned on an individual such that the opening is positioned adjacent to a female urethral opening or male urethral opening (i.e., on a penis). The individual may discharge one or more bodily fluids, such as urine, blood, or sweat. The bodily fluids may flow into the chamber and be received into the porous material. The bodily fluids may be removed from the chamber via the fluid outlet. In an embodiment, a suction may be applied to the chamber from a vacuum source which removes the bodily fluids from the chamber.

The shape memory material disposed in the shape memory lumen that is retained therein by the stop plug and end plug allows the fluid collection assembly to be bent, molded, or otherwise shaped into a selected configuration. The shape memory material disposed in the shape memory lumen that is retained therein by the stop plug and end plug allows the shape memory material to move within the shape memory lumen in a longitudinal direction which provides excellent malleability compared to shape memory material that is longitudinally fixed in a shape memory lumen. The end plug and stop plug also prevent corrosion of, or contamination from, the shape memory material by sealing the shape memory material within the shape memory lumen. Such a configuration also prevents wearer's from feeling the shape memory material by disposing it inside of the conduit.

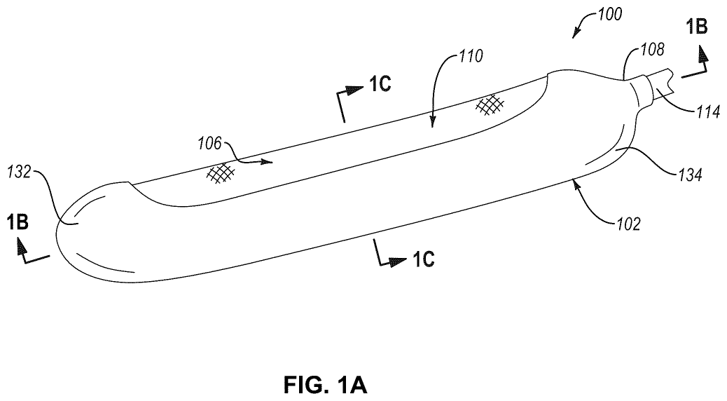

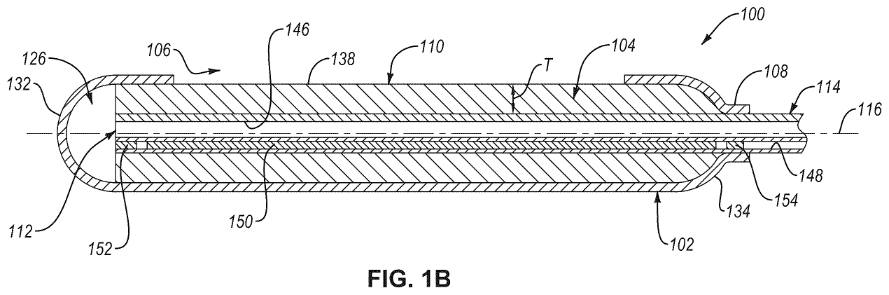

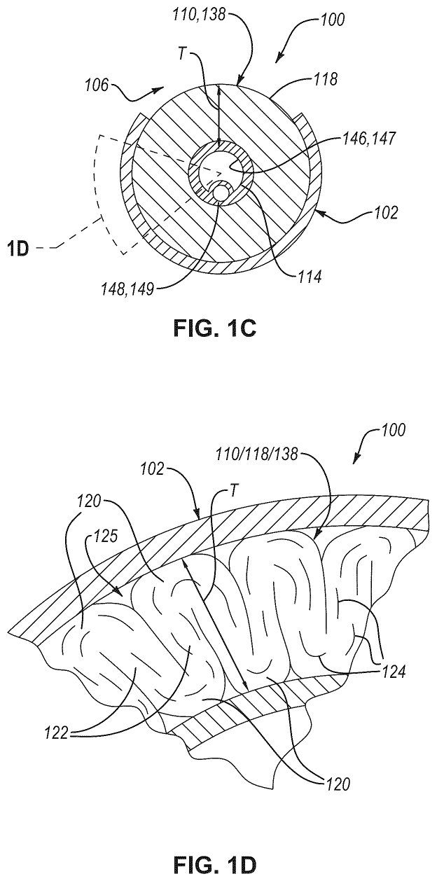

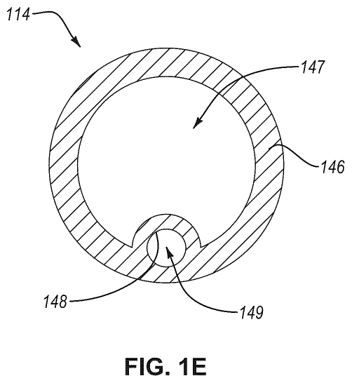

FIG. 1A is an isometric view of a fluid collection assembly 100, according to an embodiment. FIGS. 1B and 1C are cross-sectional schematics of the fluid collection assembly 100 taken along planes 1B-1B and IC-IC, respectively, shown in FIG. 1A. The fluid collection assembly is an example of a fluid collection assembly configured to receive bodily fluids from a urethral opening. The fluid collection assembly 100 includes a fluid impermeable layer 102. The fluid impermeable layer 102 at least defines a chamber 104, at least one opening 106, and a fluid outlet 108. The fluid collection assembly 100 also includes at least one porous material 110 disposed in the chamber 104 that extends across the opening 106. The porous material 110 includes at least one porous body material 138. The fluid collection assembly 100 also includes a conduit 114 having a multi-lumen configuration.

The fluid impermeable layer 102 at least partially defines a chamber 104 (e.g., interior region) and an opening 106. The fluid impermeable layer 102 temporarily stores the bodily fluids in the chamber 104. The fluid impermeable layer 102 may be formed of any suitable fluid impermeable material(s), such as a fluid impermeable polymer (e.g., silicone, polypropylene, polyethylene, polyethylene terephthalate, neoprene, a polycarbonate, etc.), a metal film, natural rubber, another suitable material, any other fluid impermeable material disclosed herein, or combinations thereof. As such, the fluid impermeable layer 102 substantially prevents the bodily fluids from passing through the fluid impermeable layer 102. In an example, the fluid impermeable layer 102 may be air permeable and fluid impermeable. In such an example, the fluid impermeable layer 102 may be formed of a hydrophobic material that defines a plurality of pores. At least one or more portions of at least an outer surface of the fluid impermeable layer 102 may be formed from a soft and/or smooth material, thereby reducing chaffing on a wearer or user of the assembly.

The opening 106 provides an ingress route for bodily fluids to enter the chamber 104. The opening 106 may be defined by the fluid impermeable layer 102 such as by an inner edge of the fluid impermeable layer 102. For example, the opening 106 is formed in and extends through the fluid impermeable layer 102 thereby enabling bodily fluids to enter the chamber 104 from outside of the fluid collection assembly 100.

In some examples, the fluid impermeable layer 102 may define a fluid outlet 108 sized to receive the conduit 114. The at least one conduit 114 may be disposed in the chamber 104 via the fluid outlet 108. The fluid outlet 108 may be sized and shaped to form an at least substantially fluid tight seal against the conduit 114 (e.g., drainage tube) thereby substantially preventing the bodily fluids from escaping the chamber 104.

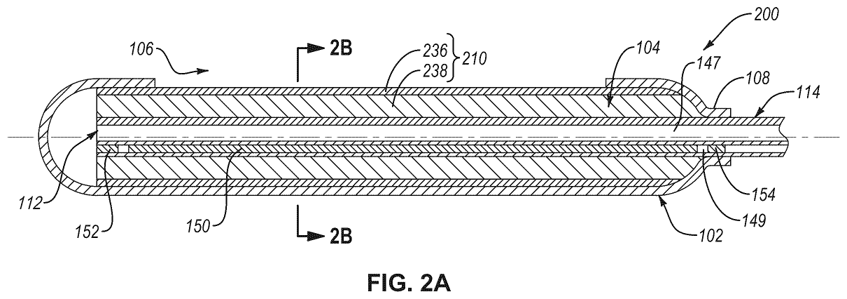

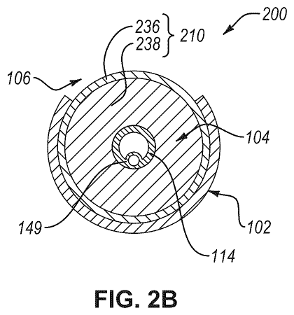

As previously discussed, the fluid collection assembly 100 includes porous material 110 disposed in the chamber 104. The porous material 110 may cover at least a portion (e.g., all) of the opening 106. The at least one porous material 110 may include one or more of a foam, spun fibers, a vertical nonwoven material, a woven material, a quilted material, or the like. The at least one porous material 110 may include a porous body material 138 and, optionally, a porous membrane material disposed over the porous body material (FIGS. 2A and 2B).

The at least one porous material 110 may be formed from any suitable natural material (e.g., fibers, fabric, foam) or synthetic material (e.g., fibers, fabric, foam). In some embodiments, the at least one porous material 110 may include an open cell foam, a carded web, spun fibers (e.g., spun nylon fibers), nonwoven fibers, woven fibers, or the like. For example, the porous body material 138 may include an open cell foam or a vertically nonwoven material. In an embodiment, the at least one porous material 110 (e.g., porous body material 138) may be formed from synthetic fibers or foam, such a polymer fibers or foam. Examples of synthetic fibers or foam includes polyester, polyethylene, polypropylene, polyurethane (e.g. viscoelastic PU), latex, silicone, nylon, or the like. Some embodiments, the at least one porous material 110 may be formed from natural fibers which may be more sustainable and biodegradable than the synthetic fibers. Examples of natural fibers includes cellulose, cotton, bamboo, wool, or the like.

In some embodiments, an open-cell foam may be used for the porous material 110 (e.g., porous body material 138). The open-cell foam may have a density and porosity selected to provide a desired amount and rate of fluid transport therethrough. For example, the open-cell foam may have a density of at least 40 kg/m³, such as about 40 kg/m³ to about 500 kg/m³, about 50 kg/m³ to about 400 kg/m³, about 50 kg/m³ to about 200 kg/m³, about 200 kg/m³ to about 400 kg/m³, or less than 500 kg/m³. The open-cell foam may exhibit a porosity of at least about 15 pores per inch (PPI), such as about 20 PPI to about 120 PPI, about 20 PPI to about 50 PPI, about 50 PPI to about 100 PPI, or less than about 120 PPI. The material of the open-cell foam may be selected to have desired surface properties (e.g., hydrophilicity or hydrophobicity) and structural properties (e.g., bending stiffness or the like). As explained in more detail below, the open-cell foam may include any of a number of different materials, such as rubber, one or more polymers, or the like.

Generally, the average person discharges urine at a rate of about 6 ml/s to about 50 ml/s, such as at a rate of about 10 ml/s to about 25 ml/s. The rate at which the person urinate may vary, such as based on the size of the person and the age of the person. The porous material 110 may be selected to capture and transport the bodily fluids at a rate that is comparable to the rate at which the individual discharged bodily fluids to prevent leaks. For example, the at least one porous material 110 may be selected to capture and transport the bodily fluids at a rate that is greater than about 6 ml/s, greater than about 10 ml/s, greater than about 30 ml/s, about 6 ml/s to about 50 ml/s, about 6 ml/s to about 20 ml/s, about 20 ml/s to about 40 ml/s, about 6 ml/s to about 15 ml/s, about 15 ml/s to about 25 ml/s, less than about 50 ml/s, or less than about 30 ml/s.

In some embodiments, at least one porous material 110 (e.g., porous body material 138) may be configured to wick and/or otherwise allow transport of any bodily fluids away from the opening 106, thereby preventing the bodily fluids from escaping the chamber 104. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the at least one porous material 110. Put another way, substantially no absorption or solubility of the bodily fluids into the at least one porous material 110 material may take place after the at least one porous material 110 is exposed to the bodily fluids and removed from the bodily fluids for a time. While no absorption or solubility is desired, the term “substantially no absorption” may allow for nominal amounts of absorption and/or solubility of the bodily fluids into the at least one porous material 110 (e.g., absorbency), such as less than about 30 wt % of the dry weight of the at least one porous material 110, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the at least one porous material 110. In an embodiment, the at least one porous material 110 may include at least one absorbent or adsorbent material.

In an embodiment, the at least one porous material 110 may be hydrophobic. The at least one porous material 110 may be hydrophobic when the at least one porous material 110 exhibits a contact angle with water that is about 90° to about 120°, about 105° to about 135°, about 120° to about 150°, about 135° to about 165°, or greater than 150°. The hydrophobic at least one porous material 110 may more quickly transport the bodily fluids received thereby than if the at least one porous material 110 is hydrophilic.

Notwithstanding the foregoing, in an embodiment, the at least one porous material 110 may by hydrophilic. The hydrophilicity of the at least one porous material 110 may cause the at least one porous material 110 to quickly capture bodily fluids therein thereby preventing or at least inhibiting leakage of bodily fluids caused by a large discharge of bodily fluids over a short period of time. The at least one porous material 110 may be hydrophilic when the at least one porous material 110 material exhibits a contact angle with water (a major constituent of bodily fluids) that is about 0° to about 90°, about 0° to about 15°, about 15° to about 30°, about 30° to about 45°, about 45° to about 60°, about 60° to about 90°, about 10° to about 40°, about 40° to about 80°, less than about 90°, less than about 60°, or less than about 30°. Generally, increasing the hydrophilicity of the at least one porous material 110 (i.e., decreasing the contact angle between the vertical nonwoven material and water) increases the quantity of bodily fluids that the at least one porous material 110 may receive over a certain period of time. However, increasing the hydrophilicity of the at least one porous material 110 may increase the quantity of bodily fluids that are retained in the at least one porous material 110 after the at least one porous material 110 receives the bodily fluids. As such, the hydrophilicity of the at least one porous material 110 may be selected based on balancing the need to receive bodily fluids quickly while also keeping the at least one porous material 110 dry. For example, a fluid collection assembly 100 configured to be used with an individual with a large bladder for short periods of time may include a at least one porous material 110 exhibiting a hydrophilicity that is greater than an least one porous material 110 of a fluid collection assembly 100 configured to be used with an individual with an average to small sized bladder for long period of time. It is noted that materials of at least some conventional fluid collection assemblies are selected to be hydrophobic to improve the fluid transport thereof. However, it has been unexpectedly found that vertical nonwoven materials exhibit quick fluid transport even when the vertical nonwoven materials are hydrophilic.

In an embodiment, the hydrophobicity or hydrophilicity of the at least one porous material 110 may be an inherent property of the material(s) (e.g., fibers) used to form the at least one porous material 110. In an embodiment, the hydrophobicity or hydrophilicity of the at least one porous material 110 may be changed by at least one of impurities or functional groups added to the at least one porous material 110, otherwise treating the at least one porous material 110, or coating the at least one porous material 110 with a material that exhibits a hydrophobicity or hydrophilicity that is different than the at least one porous material 110.

FIG. 1D is a cross-sectional schematic of a portion of the fluid collection assembly 100 taken from the box illustrated in FIG. 1C, according to an embodiment. As shown, the at least one porous material 110 may include a vertical nonwoven material. The vertical nonwoven material is formed from a nonwoven web 118 that is folded. The folded nonwoven web 118 may include a plurality of folded portions 120 and a plurality of intermediate portions 122 extending between the folded portions 120. The folded nonwoven web 118 may include an outer surface adjacent to the fluid impermeable layer 102 and an opposing inner surface (e.g., defining a bore that received the conduit 114). The folded portion 120 may extend generally parallel to the outer and inner surfaces of folded nonwoven web 118. The intermediate portions 122 may extend between the outer and inner surfaces of the folded nonwoven web 118. In an embodiment, the folded nonwoven web 118 may be positioned in the chamber 104 such that the folded portions 120 extend generally parallel to a longitudinal (e.g., central) axis 116 of the fluid collection assembly 100 (e.g., generally parallel to a longitudinal axis of the porous material 110) and/or extend circumferentially when the porous material 110 exhibits a generally cylindrical shape. The folded nonwoven web 118 may be positioned in the chamber 104 such that the intermediate portions 122 extend generally parallel to the longitudinal axis 116 of the fluid collection assembly 100 (e.g., generally parallel to a longitudinal axis of the porous material 110) and/or extend radially when the porous material 110 exhibits a generally cylindrical shape.

The vertical nonwoven material includes a plurality of fibers 124. In an embodiment, the nonwoven web 118 that forms the vertical nonwoven material may include a plurality of generally oriented fibers 124. The generally oriented fibers 124 may improve the ability of the vertical nonwoven material to capture bodily fluids and transport bodily fluids. The generally oriented fibers 124 may also improve the mechanical properties of the vertical nonwoven material. As used herein, the fibers 124 are “generally aligned” when a certain percentage of the fibers 124 are substantially parallel to each other. The certain percentage of the fibers 124 refers to at least about 70% of the fibers 124, more preferably at least about 80% of the fibers 124, more preferable 90% of the fibers 124, and even more preferably at least about 95% of the fibers 124. The fibers 124 are generally parallel to each other when the certain percentage of fibers 124 are parallel to each other ±30° more preferable ±20°, more preferably ±10°, or even more preferably ±5°.

The nonwoven web 118 may be disposed in the chamber 104 such that the fibers 124 of the folded portions 120 are generally oriented circumferentially and the fibers 124 of the intermediate portions 122 are generally oriented radially. Not wishing to be bound to theory, the circumferentially orientation of the fibers 124 of the folded portions 120 may cause the bodily fluids received by the vertical nonwoven material to initially preferentially disperse circumferentially and the radial orientation of the fibers 124 of the intermediate portions may cause the bodily fluids to initially preferentially disperse radially into the porous material 110. Causing the bodily fluids to initially disperse circumferentially and radially quickly disperses the bodily fluids throughout a large volume of the vertical nonwoven material thereby allowing the vertical nonwoven material to quickly capture and transport the bodily fluids. It is noted that the fibers 124 do not inhibit flow of the bodily fluids in a direction that is generally parallel to the longitudinal axis 116, especially after the fibers 124 are wetted. Further, dispersing the bodily fluids throughout the vertical nonwoven material increases the surface area of any bodily fluids that may remain in the vertical nonwoven material after removing the bodily fluids from the porous material 110. The large surface area facilitates evaporation of the remaining bodily fluids with the air flow through the porous material 110. In an embodiment, the fibers 124 are randomly oriented or may be oriented differently than what is shown in FIG. 1D.

In an embodiment, as illustrated, folding the nonwoven web 118 may cause the formation of gaps 125 that extending generally parallel to the longitudinal axis 116. The gaps 125 may facilitate fluid flow in a direction that is generally parallel to the longitudinal axis 116. However, the nonwoven web 118 may be folded or compressed by the fluid impermeable layer 102 to minimize the size of the gaps 125 to prevent pooling of the bodily fluids in the chamber 104. For example, the nonwoven web 118 may be folded or compressed by the fluid impermeable layer 102 to cause the gaps 125 to exhibit a dimension measured perpendicular to the longitudinal axis 116 that is less than about 1 mm, less than about 0.75 mm, less than about 0.5 mm, or less than about 0.25 mm.

As previously discussed, the vertical nonwoven material may be formed from at least one nonwoven web 118 that is folded. The vertical nonwoven material may be formed from any suitable nonwoven web. In an embodiment, the nonwoven web includes at least one carded web. The carded web includes a plurality of fibers 124 that may be generally oriented in the same direction. The generally same orientation of the fibers 124 of the carded web cause the carded web to be anisotropic. For example, the strength of the carded web is greatest when a force applied thereto is generally parallel to the fibers 124 but the strength of the carded web decreases as the force applied thereto becomes more oblique or perpendicular to the orientation of the fibers 124. As such, the carded web may need to be positioned in the chamber 104 to mitigate forces being applied to the carded web that are not generally parallel to the orientation of the fibers 124 or requires addition binding between the fibers 124 (e.g., heat or chemical) to prevent unsatisfactory wear of the carded web.

In an embodiment, the nonwoven web 118 may include at least one needle punched web. The needle punched web may be formed from a sheet including a plurality of fibers 124. The sheet may include a plurality of randomly oriented fibers 124 (e.g., the fibers 124 are generally parallel to and randomly oriented in the plane), or generally oriented fibers 124 (e.g., a carded web) since the orientation of the fibers 124 may better facilitate flow of the bodily fluids therethrough. A plurality of needles (e.g., a plurality of barbed needles) are inserted into the sheet in a direction that is generally parallel to a thickness of the sheet which causes some of the fibers 124 to become entangled and interlocked.

In an embodiment, the nonwoven web 118 may include at least one air laid web. The air laid web may exhibit a plurality of randomly oriented fibers 124. The plurality of random fibers 124 may exhibit a length that is sufficiently large that the fibers 124 become entangled and do not need be bounded together or the fibers 124 may be bonded. Due to the random orientation of the fibers 124, the air laid web tends to be isotropic and exhibit a high porosity. Similar, due to the random orientation of the fibers 124, the air laid web may exhibit a high loft. The air laid web may be formed from fibers 124 that cannot be carded (e.g., short fibers).

In an embodiment, the nonwoven web 118 may include at least one spunlaced web. The spunlaced web is formed by providing a sheet that includes randomly oriented fibers 124 or a carded web. High pressure water jets that are generally parallel to the thickness of the sheet are directed towards the sheet. Similar to the needle punched web, the high pressure jets of water cause some of the fibers 124 to migrate from an exterior of the sheet to an interior thereof to form columns. Thus, the spunlaced web may function similar to the needle punched web, namely that the spunlaced web may be more isotropic than the carded web and includes divots.

While carded web, needle punched web, the air laid web, and the spunlaced web are the preferred nonwoven webs to be included in the vertical nonwoven material, the vertical nonwoven material may include one or more nonwoven webs other than the carded web, needle punched web, the air laid web, and the spunlaced web. For example, the vertical nonwoven material may include a wet laid web, a spunbond nonwoven web, or a meltblown nonwoven web.

The folded nonwoven web 118 may be formed from a sheet. When resting the sheet on a horizontal planar surface, the folded portions 120 may extend parallel to the horizontal planar surface and the intermediate portions 122 may extend vertically from the horizontal planar surface. The folded nonwoven web 118 may then be rolled to form the cylindrical folded nonwoven web 118.

In some embodiments, the vertical nonwoven material may include a material there does not have fibers. For example, a foam body may be utilized instead of a nonwoven web 118. The foam body may be sized, shaped, and folded similarly or identically to the folded nonwoven web 118 to form the porous body of the porous material 110. For example, the foam body may include an open cell foam body folded to have the plurality of folded portions 120 and the plurality of intermediate portions 122 extending between the folded portions 120 to form the vertical nonwoven material. In such examples, the foam body may include a foam of any of the polymers disclosed herein, such as polyurethane, polyethylene, polyethylene terephthalate, polyether, or the like. Bodily fluids may move through the open cell structure of the foam toward the reservoir or vacuum force (e.g., inlet of conduit 114).

Suitable vertical nonwoven materials and their properties for use in the porous material 110 are disclosed in International Patent Application No. PCT/US2022/042719 filed on 7 Sep. 2022, and U.S. Provisional Patent Application No. 63/241,575 filed on 8 Sep. 2021, the disclosure of each of which is incorporated herein, in its entirety, by this reference for any purpose.

The porous material 110 may exhibit a thickness T that is greater than about 1 mm, such as in ranges of 1 mm to about 30 mm, about 1 mm to about 10 mm, about 10 mm to about 20 mm, about 20 mm to about 30 mm, about 5 mm to about 15 mm, about 15 mm to about 25 mm, less than about 30 mm, or less than about 20 mm. Increasing the thickness T of the porous material 110 generally increases the volume of bodily fluids that may be temporarily stored in the porous material 110, and allows greater flexibility in selecting the density and basis weight of the porous material 110. However, the thickness T of the porous material 110 may be limited by the size and functionality of the fluid collection assembly 100. For example, the thickness T of the porous material 110 may be selected such that the porous material 110 may be disposed in the chamber 104, along with any other components that may also be disposed in the chamber 104, such as porous membrane material 236 (FIG. 2B), or a conduit 114.

The rate at which the porous material 110 captures and transports the bodily fluids may depend on a number of factors. For example, the rate at which a vertical nonwoven material captures and transports the bodily fluids may depend inversely on the density and weight basis of the vertical nonwoven material, wherein increasing the density and/or weight basis of the vertical nonwoven material may decrease the rate at which the vertical nonwoven material captures and transports the bodily fluids and vice versa. In an example, the rate at which the porous material 110 captures and transports the bodily fluids may depend on the material (e.g., hydrophilicity of the material) that forms the porous material 110. The rate at which the porous material 110 captures and transports the bodily fluids may increase with increasing thickness T since increasing the thickness T increases the cross-sectional area through which the bodily fluids may flow.

Referring back to FIGS. 1A-1C, in an embodiment, as illustrated, the porous material 110 may only or substantially only include the porous body material 138. In such an embodiment, the porous body material 138 may define a bore that is configured to receive the conduit 114 and the porous body material 138 extends from the bore to the fluid impermeable layer 102. When the porous material 110 includes only or substantially only the porous body material 138, all of the porous material 110 is able to quickly capture and transport the bodily fluids. As explained in more detail below, the porous material 110 may include at least one additional material, such as porous membrane material, even though such additional material may decrease at least one of the ability of the porous material 110 to capture and/or transport the bodily fluids.

The fluid collection assembly 100 may include a sump therein to collect bodily fluids. The sump may be an occupied or unoccupied portion of the chamber 104. The sump may be a portion within the chamber 104 at or near where the inlet of the conduit 114 is located. The porous material 110 may at least substantially completely fill the portions of the chamber 104 that are not occupied by the conduit 114. In some examples, the porous material 110 may not substantially completely fill the portions of the chamber 104 that are not occupied by the conduit 114. In such an example, the fluid collection assembly 100 includes a reservoir 126 (e.g., sump) disposed in the chamber 104.

As depicted, the reservoir 126 (sump) may be a substantially unoccupied portion of the chamber 104. The reservoir 126 may be defined between the fluid impermeable layer 102 and porous material 110. The bodily fluids that are in the chamber 104 may flow through the porous material 110 to the reservoir 126. The reservoir 126 may retain of the bodily fluids therein. The fluid impermeable layer 102 may retain the bodily fluids in the reservoir 126. While depicted in the distal end region 132, the reservoir 126 may be located in any portion of the chamber 104 such as the proximal end region 134. The reservoir 126 may be located in a portion of the chamber 104 that is designed to be located in a gravimetrically low point of the fluid collection assembly when the fluid collection assembly 100 is worn.

While depicted as portions of the chamber 104 not occupied by the porous material 110, the reservoir 126 or sump may be occupied by the porous material 110 and still function as a reservoir or sump. For example, the porous material 110 may fill substantially of the chamber 104 not occupied by the conduit 114 and the reservoir 126 may be a distal end region of the chamber or any other region of the chamber 104 configured to retain the bodily fluid therein while or prior to being removed via the conduit 114.

In some examples (not shown), the fluid collection assembly 100 may include multiple reservoirs, such as a first reservoir that is located at the portion of the chamber 104 closest to the inlet of the conduit 114 (e.g., distal end region 132) and a second reservoir that is located at the portion of the of the chamber 104 that is at or near proximal end region 134). In another example, the porous material 110 is spaced from at least a portion of the conduit 114, and the reservoir 126 may be the space between the porous material 110 and the conduit 114.

FIG. 1E is a cross-sectional view of the conduit 114, according to an embodiment. Referring to FIGS. 1B, 1C and 1E, the conduit 114 includes a fluid lumen 147 and a shape memory lumen 149 therein. The conduit 114 includes an outer wall 146 and an inner wall 148 at least partially defining the fluid lumen 147 and shape memory lumen 149, respectively. The outer wall 146 and the inner wall 148 of the conduit 114 may be constructed of a polymer, such as silicone, polyvinyl chloride (PVC), polyethylene, thermoplastic polyurethane, a thermoplastic elastomer (e.g., a synthetic rubber), or the like. The conduit 114 may have a Shore A hardness of at least 40, such as about 40 to about 95, about 50 to about 95, about 40 to about 60, about 60 to about 80, about 75 to about 95, less than 95, or less than 75. The conduit 114 may have a length of at least a meter, such as about 1 m to about 5 m, about 1 m to about 3 m, about 2 m to about 4 m, or less than 5 m.

The conduit 114 may be at least partially disposed in the chamber 104. The conduit 114 may be used to remove the bodily fluids from the chamber 104. The conduit 114 includes at least one wall (e.g., outer wall 146) defining an inlet 112, an outlet (not shown) downstream from the inlet 112, and a passageway therebetween.

The outlet of the conduit 114 may be operably coupled to a vacuum source, such as a vacuum pump for withdrawing fluid from the chamber 104 through the conduit 114. For example, the conduit 114 may extend into the fluid impermeable layer 102 from the proximal end region 134 and may extend to the distal end region 132 to a point proximate to the reservoir 126 therein such that the inlet 112 is in fluid communication with the reservoir 126. The conduit 114 fluidly couples the chamber 104 with the fluid storage container (not shown) or the vacuum source (not shown). In some embodiments, the inlet 112 may be disposed at or near the fluid impermeable layer 102 in the distal end region 132.

The conduit 114 may extend through a bore in the porous material 110. In an embodiment, the conduit 114 extends from the fluid outlet 108, through the bore, to a location that is proximate to the reservoir 126. In such an embodiment, the inlet 112 may not extend into the reservoir 126 and, instead, the inlet 112 may be disposed within the porous material 110 or at a terminal end thereof. For example, an end of the conduit 114 may be coextensive with or recessed within the porous material 110. In an embodiment, the conduit 114 is at least partially disposed in the reservoir 126 and the inlet 112 may be extended into or be positioned in the reservoir 126. In an embodiment, the inlet 112 may be positioned aft of the reservoir 126. The bodily fluids collected in the fluid collection assembly 100 may be removed from the chamber 104 via the conduit 114.

Locating the inlet 112 at or near a location expected to be the gravimetrically low point of the chamber 104 when worn by an individual enables the conduit 114 to receive more of the bodily fluids than if inlet 112 was located elsewhere and reduce the likelihood of pooling (e.g., pooling of the bodily fluids may cause microbe growth and foul odors). For instance, the bodily fluids in the porous material 110 may flow in any direction due to capillary forces. However, the bodily fluids may exhibit a preference to flow in the direction of gravity, especially when at least a portion of the porous material 110 is saturated with the bodily fluids. Accordingly, one or more of the inlet 112 or the reservoir 126 may be located in the fluid collection assembly 100 in a position expected to be the gravimetrically low point in the fluid collection assembly 100 when worn by an individual, such as the distal end region 132.

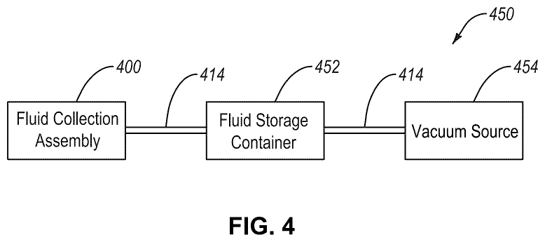

The inlet 112 and the outlet of the conduit 114 are configured to fluidly couple (e.g., directly or indirectly) the vacuum source (not shown) to the chamber 104 (e.g., the reservoir 126). As the vacuum source (FIG. 4) applies a vacuum/suction in the conduit 114, the bodily fluids in the chamber 104 (e.g., at the distal end region 132 such as in the reservoir 126) may be drawn into the inlet 112 and out of the fluid collection assembly 100 via the conduit 114. In some examples, the conduit 114 may be frosted or opaque (e.g., black) to obscure visibility of the bodily fluids therein.

As previously discussed, the conduit 114 may be configured to be at least insertable into the chamber 104. In an example, the conduit 114 may be positioned in the chamber 104 such that a terminal end of the conduit 114 is spaced from the fluid impermeable layer 102 or other components of the fluid collection assembly 100 that may at least partially obstruct or block the inlet 112. Further, the inlet 112 of the conduit 114 may be offset relative to a terminal end of the porous material 110 such that the inlet 112 is closer to the proximal end region 134 of the fluid collection assembly 100 than the terminal end of the porous material 110. Offsetting the inlet 112 in such a manner relative to the terminal end of the porous material 110 allows the inlet 112 to receive bodily fluids directly from the porous material 110 and, due to hydrogen bonding, pulls more bodily fluids from the porous material 110 into the conduit 114.

The multiple lumen configuration may include the fluid lumen 147 for transporting fluids and at least one shape memory lumen 149 for containing a shape memory material 150 therein.

The fluid lumen 147 is sized and shaped to allow bodily fluids to flow therethrough. As shown in FIG. 1E, the fluid lumen 147 may be a larger than the shape memory lumen 149. For example, the fluid lumen 147 may have an inside diameter (or major dimension) of at least about 0.1 inches (2.5 mm), such as about 0.1 inches to about 0.5 inches (12.7 mm), about 0.15 inches (3.8 mm) to about 0.35 inches (8.9 mm), about 0.25 inches (6.3 mm) to about 0.4 inches (10.2 mm), or less than about 0.5 inches.

The shape memory lumen 149 is sized and shaped to accommodate a shape memory material 150 therein. For example, the shape memory lumen 149 may have an inside diameter (or major dimension) of at least about 0.03 inches (0.76 mm), such as about 0.03 inches to about 0.1 inches (2.5 mm), about 0.03 inches to about 0.05 inches (1.3 mm), about 0.05 inches to about 0.07 inches (1.8 mm), or less than about 0.1 inches. While the shape memory lumen 149 is depicted as being positioned in the gravimetrically low point of the conduit 114 (e.g., in the bottom of the fluid lumen 147), the shape memory lumen may be disposed at any point in the fluid lumen 147, such as at the lateral sides (e.g., 3 o'clock or 9 o'clock) or the top (e.g., 12 o'clock position).

Referring back to FIGS. 1B and 1C, the shape memory lumen 149 may contain the shape memory material 150 in a discrete portion of the longitudinal length of the conduit 114, such as a portion of the shape memory lumen 149 that is within the longitudinal length of the fluid impermeable layer 102. The shape memory material 150 may include an element configured to allow the shape of the fluid collection assemblies to be controllably changed and/or maintain a selected shape.

The at least one shape memory material 150 may prevent or at least inhibit bodily fluids leaking from the fluid collection assembly 100. For example, bodily fluids may leak from the fluid collection assembly because, initially, the fluid collection assembly 100 may exhibit a poor fit with the anatomy of a wearer (e.g., user) about the urethral opening. The poor fit may cause gaps to be present between the porous material 110 and the region about the urethral opening. These gaps may provide locations through which the bodily fluids may flow without being received by the porous material 110 and/or locations at which bodily fluids may leave the porous material 110. To minimize or eliminate the gaps, the fluid collection assembly 100 includes the shape memory material 150 disposed in the shape memory lumen 149. The shape memory material 150 is configured to be manipulated (e.g., bent or otherwise shaped) which, in turn, causes the fluid collection assembly 300 to exhibit a shape that matches the anatomical shape of the patient and to conform to the shape of the anatomy of the wearer about the urethral opening (e.g., vaginal region). In other words, the shape memory material 150 enables a more anatomically precise fit for the fluid collection assembly 100 with the region about the urethral opening than conventional fluid collection devices.

The shape memory material 150 is sized, shaped, and positioned in the fluid collection assembly 100 to cause at least a portion of the fluid collection assembly 100 to retain a selected shape (e.g., geometric configuration). The shape memory material 150 is configured to be bent, shaped, or otherwise deformed (hereafter collectively referred to as “shape,” “shaped,” or “shaping”). The shape memory material 150 may be configured to be shaped along an entire length thereof. Allowing the shape memory material 150 to be shaped along the entire length thereof may allow the fluid collection assembly 100 to exhibit a shape that substantially corresponds to the anatomical features of the patient. For example, the shape memory material 150 may exhibit a first (e.g., initial) shape and the fluid collection assembly 100 may exhibit the first configuration (i.e., a generally linear shape) when the shape memory material 150 exhibits the first shape. The shape memory material 150 may be manipulated to exhibit a second shape that is different than the first shape and the fluid collection assembly 100 may exhibit the second configuration (e.g., a generally curved cylindrical shape) when the shape memory material 150 exhibits the second shape. The second configuration of the fluid collection assembly 100 may better correspond to the shape of the region about the urethral opening than the first configuration.

The shape memory material 150 may include a shape memory polymer or a metal (e.g., shape memory metal). Generally, the shape memory material 150 is composed to adopt an intermediate or permanent shape in response to a stimuli. For example, the shape memory material 150 may exhibit a first (e.g., initial) shape and may be switched from the first shape to a second shape by an external stimuli to adopt a second shape that is different from the first shape. The shape memory material 150 may also be switched from the second shape back to the first shape or a third shape that is different than the first and second shapes in response to the stimuli.

The stimuli may include an external physical force (e.g., bending force), heat, electrical bias, or a magnetic field. While the term “shape memory” is used to describe some of the “shape memory materials” herein, it should be understood that, in some examples, the material modified by the term “shape memory” may not necessarily need to return to a preselected shape upon application of a stimuli, as understood as the classical definition of the “shape memory material.” Rather, at least some of the shape memory materials disclosed herein may simply hold a selected shape when bent, set, or cured into a specific shape and/or when cooled in a specific shape, regardless of the stimuli applied thereto after. The shape memory materials may be returned to the original shape or changed to a new shape by application of the stimuli. For example, a metal wire bent to a first shape may be utilized as the shape memory material 150, whereinafter the metal wire may be modified to a second shape via physical force applied thereto or via heating. However, in some embodiments, the shape memory material 150 may exhibit a selected shape, as discussed above and application of the stimuli may cause the shape memory material to deform (e.g., elastically deform or bend) into an intermediate shape. In such embodiments, the shape memory material 150 may return to the first initial shape upon removal of the stimuli such that the shape memory material 150 does not maintain the intermediate shape.

The shape memory material 150 is distinct (e.g., may move independently) from the conduit 114. The shape memory material 150 may include a rod, a wire, a cable, or other structure disposed in the shape memory lumen 149. The shape memory material 150 may have a cylindrical shape, an extruded ovoid shape, or a polygonal extruded shape. The shape memory material 150 may be sized to fit within the shape memory lumen 149. For example, the shape memory material 150 may have a diameter (or major dimension) that is at least about 0.03 inches, such as about 0.03 inches to about 0.1 inches, about 0.03 inches to about 0.05 inches, about 0.05 inches to about 0.07 inches, less than about 0.15 inches, or less than about 0.1 inches. In some embodiments, the shape memory material 150 may include a 12 gauge (2.05 mm) to 20 gauge (0.81 mm) wire, such as a 14 gauge (1.63 mm) wire. The shape memory material 150 may be smaller than the shape memory lumen 149, such as at least 0.001 inches (25.4 μm) smaller, 0.001 inches to 0.1 inches, 0.001 inches to 0.01 inches (254 μm), 0.01 inches to 0.04 inches, 0.04 inches to 0.1 inches smaller than the shape memory lumen 149. Such examples provide a selected fit between the shape memory material 150 and the shape memory lumen 149, such as a slip fit, a sliding fit, a running fit, a loose fit, or the like that allow the the shape memory material 150 to move (e.g., longitudinally) with respect to the shape memory lumen 149.

In some examples, the shape memory material 150 may be slightly larger than the shape memory lumen 149, such as 0.001 inches larger to 0.01 inches larger. Such examples may provide a tight fit between the shape memory material 150 and the shape memory lumen 149 such that the shape memory material 150 does not move with respect to the shape memory lumen 149.

In an embodiment, the shape memory material 150 may include metal, such as an elemental metal, an alloy, or shape memory alloy. Suitable shape memory metals may include aluminum, silver, copper, iron, nickel, zinc, tin, beryllium, or the like. Suitable shape memory alloys may include alloys of any of the shape memory metals disclosed herein such as standard steels, stainless steel, carbon alloy steel, heat treated steel, galvanized steel, aluminum alloys, nickel alloys, nickel-titanium alloys (e.g., Nitinol, Ni—Ti—Cu, Ni—Ti, Co, or the like), copper, copper-based alloys (e.g., brass, Cu—Zn—Al, Cu—Al—Ni, Cu—Al—Sn, or the like), Co—Cr—Ni—Mo alloys (e.g., Elgiloy® or the like), or any other alloy having shape memory characteristics. As explained above, the shape memory metals or alloys may merely be metals or alloys that may be shaped to a selected configuration. In some examples, the shape memory metals or alloys may return to a primary shape when an external stimuli is applied thereto. In some examples, the outer surface of the shape memory metal may be coated with a polymer, anodized, passivated, or otherwise treated to prevent corrosion. In some examples, the shape memory metal may be annealed, tempered, or otherwise heat treated. Such heat treating may reduce brittle breakage and increase ductility of the shape memory material 150.

Shape memory polymers (“SMPs”) may include polyurethane-based SMPs such as a copolymer (e.g., copolyester, polyurethane, polyetherester, etc.) including blocks of one or more of poly(ε-caprolactone), polyethyleneterephthalate (PET), polyethyleneoxide (PEO), polyethylene glycol (PEG), polystyrene, polymethylmethacrylate (PMMA), Polybutylmethacrylate (PBMA), poly(N,N-butadiene), poly(N-methyl-N-oxazoline), polytetrahydrofuran, or poly(butylene terephthalate); thermoplastic polymers such as polyether ether ketone (PEEK), nylon, acetal, polytetrafluoroethylene (PTFE), polypropylene, polyethylene, acrylonitrile butadiene styrene (ABS), polysulphone, or the like; Polynorbonene; other deformable polymers; or any other shape memory polymer.

The shape memory material 150 may be retained within the shape memory lumen 149 by one or more plugs disposed at or near the (opposite) longitudinal ends thercof. The plugs may include a stop plug 154 and an end plug 152. The plugs may be sized, shaped, and located to fit within the shape memory lumen 149 and to prevent the shape memory material 150 from moving (longitudinally) within the shape memory lumen 149 beyond the plugs. For example, the stop plug 154 and end plug 152 may be disposed at opposite ends of the shape memory material 150 in the shape memory lumen 149 to prevent longitudinal movement of the shape memory material 150 within the shape memory lumen 149.

The stop plug 154 and end plug 152 may be constructed of a resilient material, such as a polymer, an epoxy, rubber, a metal, wood, or the like. Suitable polymers may include a PVC, a polycarbonate, a polyethylene, a polypropylene, a polyacetal, polytetrafluorocthylene, an acrylic, or any of the polymers disclosed herein. For example, the stop plug 154 and end plug 152 may be constructed of a heat seal PVC. The stop plug 154 and end plug 152 may be constructed of any of the metal materials disclosed herein. In such examples, the stop plug 154 and end plug 152 may be annealed, tempered, or otherwise heat treated. The stop plug 154 and end plug 152 may be constructed of an adhesive, such as adhesive dams formed in the shape memory lumen 149. The adhesive may be a UV cured adhesive, a catalyst cured adhesive, a heat cured adhesive, or the like. The stop plug 154 and end plug 152 may have a major dimension that is sized to provide a press fit, an interference fit, a fixed fit, a similar fit, a force fit, or any other tight fit within the shape memory lumen 149. For example, the stop plug 154 and end plug 152 may have a largest outer dimension that is about 0.001 inches to 0.03 inches, about 0.001 inches to about 0.01 inches, about 0.01 inches to about 0.02 inches, or less than about 0.03 inches smaller than the inner dimension of the shape memory lumen 149. The stop plug 154 and end plug 152 may match the shape of the shape memory lumen 149, such as cylindrical, an extruded ovoid, an extruded polygonal shape, or the like. In some examples, one or more portions of the stop plug 154 and end plug 152 may have a tapered shape, such as cylindrical, rounded, or the like. The stop plug 154 and end plug 152 may have a length or lengths that is at least about 0.05 inches (0.13 cm), such as about 0.05 inches to about 0.2 inches (0.51 cm), about 0.1 inches (0.25 cm) to about 0.25 inches (0.64 cm), or less than about 0.25 inches.

The end plug 152 may (longitudinally) positioned within the shape memory lumen 149 at a point therein selected to be a distal-most point that the shape memory material 150 may extend. For example, the end plug 152 may be positioned within the shape memory lumen 149 at or adjacent to the inlet 112. The end plug 152 may be spaced from the inlet 112 by at least about 0.01 inches, about 0.01 inches to about 0.25 inches, or less than about 0.5 inches. The end plug 152 is sized, shaped, and positioned to retain the shape memory material 150 within the shape memory lumen 149 by preventing the shape memory material 150 from falling out of the distal end of the shape memory lumen 149. Accordingly, the end plug 152 is retained in place within the shape memory lumen 149 such as by fit with the shape memory lumen 149, an adhesive, or welding.

The stop plug 154 may be (longitudinally) positioned within the shape memory lumen 149 at a point therein selected to be a proximal-most point that the shape memory material 150 may extend. For example, the stop plug 154 may be disposed at a point in the shape memory lumen 149 that is within the chamber 104, within the fluid outlet 108, or even outside of the fluid outlet 108 (e.g., more proximal to the wearer or user than the fluid impermeable layer).

In some embodiments, the stop plug 154 may be disposed at a point in the shape memory lumen 149 that is outside of the fluid outlet 108 (e.g., more proximal to the wearer or user than the fluid impermeable layer 102). The stop plug 154 is sized, shaped, and positioned to retain the shape memory material 150 within the distal end region of the shape memory lumen 149 by preventing the shape memory material 150 from sliding into a more proximal region of the shape memory lumen 149 than the region within or near the fluid impermeable layer 102. Accordingly, the stop plug 154 is retained in place within the shape memory lumen 149 such as by fit with the shape memory lumen 149, an adhesive, or welding.

The space in the shape memory lumen 149 between the end plug 152 and the stop plug 154 may be longer than the shape memory material 150 to allow a selected amount of movement of the shape memory material 150 therein. For example, the space in the shape memory lumen 149 between the end plug 152 and the stop plug 154 may be at least about 0.1 inches longer than the shape memory material 150, such as about 0.1 inches to about 1 inch (2.5 cm), about 0.1 inches to about 0.3 inches (0.76 cm), 0.15 (0.38 cm) inches to 0.35 inches (0.89 cm), 0.3 inches to 0.6 inches (1.52 cm), about 0.5 inches (1.27 cm) to 1 inch, less than 1 inch, or less than 0.5 inches. Such a space allows a selected amount of longitudinal movement of the shape memory material 150 within the shape memory lumen 149. The ability of the shape memory material 150 to move within the shape memory lumen 149, provided by the space and a shape memory material 150 that is slidable within the shape memory lumen 149, allows for better bending (radial bending, kinking, or the like) of the shape memory material 150, conduit 114, and fluid collection assembly 100, than a shape memory material 150 that is fixed with respect to the shape memory lumen 149. Such improved bending is due to a reduction in tensile forces introduced during bending of the shape memory material 150 when the shape memory material 150 is not adhered to the inside surface of the shape memory lumen 149.

As noted above, the end plug 152 and stop plug 154 may be retained within the shape memory lumen 149 by an adhesive, fit (between the plugs and the lumen), or welding (e.g., melting). The stop plug 154 may have an adhesive applied thereto prior to positioning with the shape memory lumen 149. After the stop plug 154 is positioned and the adhesive cures or dries, the shape memory material 150 may be disposed in the shape memory lumen 149 so that the shape memory material can slide or move longitudinally therein. The end plug 152 may have an adhesive applied thereto prior to positioning with the shape memory lumen 149, such that a selected amount of space is provided between the plugs and the shape memory material 150.

The porous materials of fluid collection assemblies disclosed herein may include both a porous (fluid permeable) body and a porous membrane. FIG. 2A is a cross-sectional schematic of a fluid collection assembly 200, according to an embodiment. FIG. 2B is a cross-sectional schematic of the fluid collection assembly 200 taken along plane 2B-2B shown in FIG. 2A. The fluid collection assembly 200 includes a porous material 210 having a porous body material 238 and a porous membrane material 236 disposed thereover. Except as otherwise disclosed herein, the fluid collection assembly 200 may be similar or identical to any of the fluid collection assemblies disclosed herein. For example, the fluid collection assembly 200 may include a fluid impermeable layer 102 at least defining a chamber 104, at least one opening 106, and a fluid outlet 108. The fluid collection assembly 200 includes the conduit 114, having the shape memory material 150, end plug 152, and stop plug 154 therein. Also, the at least one porous body material 238 of the porous material 210 may be the same or substantially similar to any of the porous materials 110 disclosed herein, such as any of the porous body materials 138 disclosed herein (e.g., polyurethane foam, spun nylon fibers, or vertical nonwoven material). In an embodiment, the porous body material 238 may support the porous membrane material 236 disposed thereover, while in other embodiments the porous membrane material 236 may be supported by the fluid impermeable layer 102 or other component.

The porous membrane material 236 may include any suitable porous material, such as a porous sheet. In an example, the porous membrane material 236 may include gauze (e.g., a silk, linen, or cotton gauze), another soft fabric, another smooth fabric, a horizontal lapped nonwoven material, a cross lapped nonwoven material, a porous polymer (e.g., nylon, polyester, polyurethane, polyethylene, polypropylene, etc.) structure or an open cell foam (e.g., spun nylon fiber), or any other suitable porous material. The porous membrane material 236 may be formed from natural fibers which may be more sustainable and biodegradable than the synthetic fibers. Examples of natural fibers includes cellulose, cotton, bamboo, wool, or the like. In some examples, the fibers of the porous membrane material 236 (e.g., gauze) may include a blend of natural fibers and synthetic fibers. for example, the porous membrane material 236 may include bamboo fibers and polypropylene fibers. In an example, the porous membrane material 236 may include a hydrophobic material (e.g., a material exhibiting a contact angle with water that is greater than) 90°).

In an example, the porous membrane material 236 (e.g., a gauze, a vertical nonwoven material, or a quilted material) may exhibit a density, basis weight, thickness, different average fiber length, different average fiber lateral dimension, different average fiber aspect ratio, or different rate at which the porous membrane material 236 captures and transports the bodily fluids than the porous body material 238.

In some examples, the porous membrane material may include a three dimensional mesh material (3D mesh material). The 3D mesh material may include a top layer, a fiber layer, and a bottom layer. The top layer includes a mesh textile. The mesh textile may include polyester, polypropylene, nylon, cellulose, cotton, bamboo, any other materials, or combinations of any of the foregoing. The top layer may be hydrophilic. The hydrophilicity may be from the material itself or a coating applied thereto. By utilizing a hydrophilic material in the top layer, moisture is preferentially moved away from the skin of the wearer of the fluid collection assembly 200. The top layer may include pores formed in the mesh. Larger pores may provide faster incorporation of urine into the porous material below (e.g., fiber layer or porous body material 238). The fiber layer includes a plurality of fibers, such as spun plastic fibers or the like. The fiber layer may include polyester, nylon, or cellulose fibers. The fiber layer may be bound to the top layer by one or more of an adhesive, welding (e.g., melting together), or the like. The fiber layer is constructed to separate the top layer from the bottom layer and move urine from the top layer to the bottom layer without retaining the urine. The bottom layer includes a mesh textile. The mesh textile may include polyester, polypropylene, nylon, cellulose, cotton, bamboo, any other materials, or combinations of any of the foregoing. The bottom may be hydrophobic. The hydrophobicity may be from the material itself or a coating applied thereto. The bottom layer includes pores formed in the mesh. The pores in the top layer may be larger than the pores in the bottom layer. The bottom layer may be joined to the fiber layer in the same manner as the top layer.

In an embodiment, as illustrated, the porous membrane material 236 is disposed on an outer surface of the at least one porous body material 238 (e.g., between the fluid impermeable layer 102 and the porous body material 138) such that the porous membrane material 236 extends across the opening 106 and contacts the individual during use. The porous membrane material 236 may be disposed on the porous body material 238 to make the fluid collection assembly 200 more comfortable to use and/or improve capture of the bodily fluids. In an example, an individual may find direct contact between the porous body material 238 and the sensitive vaginal region of the individual uncomfortable, for instance, due to the surface roughness of a foam or fibers protruding of the porous body material 238. In such an example, the porous membrane material 236 may include a material (e.g. gauze) that is smoother or otherwise more comfortable than the porous body material 238. In an example, as previously discussed, the hydrophilicity of the porous body material 238 may be limited to facilitate removing bodily fluids therefrom. However, limiting the hydrophilicity of the porous body material 238 may limit the ability of the porous body material 238 to capture bodily fluids. As such, the porous membrane material 236 may be selected to exhibit a hydrophilicity that is greater than (i.e., a contact angle with water that is less than) the porous body material 238 which allows the porous membrane material 236 to capture bodily fluids more quickly than the porous body material 238. When the porous membrane material 236 exhibits a hydrophilicity that is greater than the porous body material 238, the porous membrane material 236 may exhibit a thickness that is significantly less than the thickness of the porous body material 238. The smaller thickness of the porous membrane material 236 decreases the volume of bodily fluids that are retained in the porous membrane material 236 that have to be evaporated by air flow through the chamber 204.

In an embodiment, the porous membrane material 236 may be positioned between the porous body material 238 and the conduit 114 instead of or in addition to being disposed on the outer surface of the porous body material 238.

In some examples, the porous membrane material may include a three dimensional mesh material (3D mesh material). The 3D mesh material may include a top layer, a fiber layer, and a bottom layer. The top layer includes a mesh textile. The mesh textile may include polyester, polypropylene, nylon, cellulose, cotton, bamboo, any other materials, or combinations of any of the foregoing. The top layer may be hydrophilic. The hydrophilicity may be from the material itself or a coating applied thereto. By utilizing a hydrophilic material in the top layer, moisture is preferentially moved away from the skin of the wearer of the fluid collection assembly 200. The top layer may include pores formed in the mesh. Larger pores may provide faster incorporation of urine into the porous material below (e.g., fiber layer or porous body material 238). The fiber layer includes a plurality of fibers, such as spun plastic fibers or the like. The fiber layer may include polyester, nylon, or cellulose fibers. The fiber layer may be bound to the top layer by one or more of an adhesive, welding (e.g., melting together), or the like. The fiber layer is constructed to separate the top layer from the bottom layer and move urine from the top layer to the bottom layer without retaining the urine. The bottom layer includes a mesh textile. The mesh textile may include polyester, polypropylene, nylon, cellulose, cotton, bamboo, any other materials, or combinations of any of the foregoing. The bottom may be hydrophobic. The hydrophobicity may be from the material itself or a coating applied thereto. The bottom layer includes pores formed in the mesh. The pores in the top layer may be larger than the pores in the bottom layer. The bottom layer may be joined to the fiber layer in the same manner as the top layer.

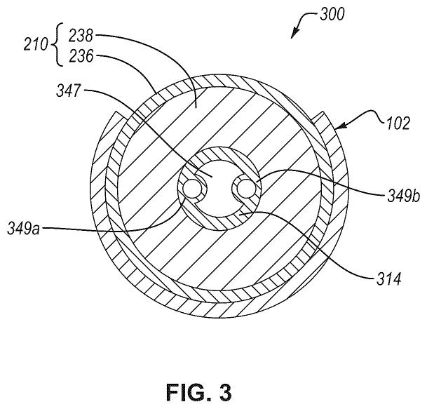

As shown in FIGS. 2A and 2B, the shape memory lumen 149 of the conduit 114 may be positioned in a gravimetrically low point of the conduit 114 (e.g., bottom of the conduit 114). Different locations and configurations of shape memory lumens and shape memory materials therein may be utilized. For example, a conduit may include multiple shape memory lumens therein. FIG. 3 is a cross-sectional view of a fluid collection assembly 300 according to an embodiment. The fluid collection assembly 300 may be similar or identical to any of the fluid collection assemblies disclosed herein, in one or more aspects. For example and as shown, the fluid collection assembly 300 includes the fluid impermeable layer 102, the porous material 210 disposed in the fluid impermeable layer 102, conduit 314 disposed in the porous material 210. The fluid collection assembly 300 may include shape memory material (FIG. 1B), end plugs (FIG. 1B) and stop plugs (FIG. 1B) therein.

The conduit 314 may be similar or identical to the conduit 114 in one or more aspects, such as size, components, material, and properties. For example, the conduit 314 may include a fluid lumen 347 and a plurality of shape memory lumens 349a and 349b therein. The fluid lumen 347 may be similar or identical to the fluid lumen 147 in one or more aspects. For example, the fluid lumen 147 may be the largest lumen on the multiple lumen conduit 314.

The plurality of shape memory lumens 349a and 349b may be similar or identical to the shape memory lumen 149 in one or more aspects, such as size, shape, or properties. The plurality of shape memory lumens may include at least 2 shape memory lumens as shown, such as 2 shape memory lumens to 20 shape memory lumens, less than 10 shape memory lumens, or less than 5 shape memory lumens.

The shape memory lumens 349a and 349b are defined by the outer walls and inner walls of the conduit 314 as described above with respect to the conduit 114. The shape memory lumens 349a and 349b may be disposed at any point in the fluid lumen 147, such as at the lateral sides (e.g., 3 o'clock or 9 o'clock) as shown, the top and bottom (e.g., 12 o'clock and 6 o'clock), both near the bottom (e.g., 5 o'clock and 7 o'clock), or any other suitable position in the fluid lumen 347.

Each of the plurality of shape memory lumens may include shape memory material, an end plug, and a stop plug therein as described with respect to the fluid collection assemblies 100 and 200. Such embodiments may provide a more rigid fluid collection assembly or allow the use of narrower shape memory materials and shape memory conduits than embodiments with a single shape memory conduit and shape memory material therein. In some embodiments, only selected ones of the plurality of shape memory lumens may include shape memory material, an end plug, and a stop plug therein.

In some examples, the conduit may include a shape memory material coextruded in the outer wall of the conduit. For example, one or more metal wires may be coextruded with the outer wall to form the conduit. In such examples, the shape memory material may include any of the shape memory materials and characteristics thereof disclosed above. Suitable coextruded conduits or drainage tubes, shape memory materials, and their properties for use in the fluid collection assemblies herein are disclosed in U.S. patent application Ser. No. 17/451,719 filed on 19 Oct. 2021, and U.S. Provisional Patent Application No. 63/094,626 filed on 21 October 2020, the disclosure of each of which is incorporated herein, in its entirety, by this reference for any purpose.

The fluid collection assemblies shown in FIGS. 1A-3 are examples of fluid collection assemblies that are particularly useful to collect bodily fluids from females (e.g., collect urine from a female urethral opening), obese males, males with buried penis, or males with a micro-penis. However, the porous materials and conduits containing shape memory materials disclosed herein may be used in fluid collection assemblies where a penis is insertable into the fluid collection assembly. Examples of male fluid collection assemblies that may include the porous materials and conduits containing shape memory materials therein, as discussed herein, are disclosed in PCT Patent Application No. PCT/US2021/039866 filed on Jun. 30, 2021 and U.S. patent application Ser. No. 16/433,773 filed on Jun. 6, 2019, the disclosure of each of which is incorporated herein, in its entirety, by this reference for any purpose.

FIG. 4 is a block diagram of a fluid collection system 450 for fluid collection, according to an embodiment. The fluid collection system 450 includes a fluid collection assembly 400, a fluid storage container 452, and a vacuum source 454. The fluid collection assembly 400 may be the same or substantially similar to any of the fluid collection assemblies disclosed herein. The fluid collection assembly 400, the fluid storage container 452, and the vacuum source 454 may be fluidly coupled to each other via one or more conduits 414. For example, fluid collection assembly 400 may be operably coupled to one or more of the fluid storage container 452 or the vacuum source 454 via the conduit 414. The bodily fluids collected in the fluid collection assembly 400 may be removed from the fluid collection assembly 400 via the conduit 414 which protrudes into the fluid collection assembly 400. For example, an inlet of the conduit 414 may extend into the fluid collection assembly 400, such as to a reservoir therein. The outlet of the conduit 414 may extend into the fluid collection assembly 400 or the vacuum source 454. Suction force may be introduced into the chamber of the fluid collection assembly 400 via the inlet of the conduit 414 responsive to suction (e.g., vacuum) force applied at the outlet of the conduit 414.

The suction force may be applied to the outlet of the conduit 414 by the vacuum source 454 either directly or indirectly. The suction force may be applied indirectly via the fluid storage container 452. For example, the outlet of the conduit 414 may be disposed within the fluid storage container 452 and an additional conduit 414 may extend from the fluid storage container 452 to the vacuum source 454. Accordingly, the vacuum source 454 may apply suction to the fluid collection assembly 400 via the fluid storage container 452. The suction force may be applied directly via the vacuum source 454. For example, the outlet of the conduit 414 may be disposed within the vacuum source 454. An additional conduit 414 may extend from the vacuum source 454 to a point outside of the fluid collection assembly 400, such as to the fluid storage container 452. In such examples, the vacuum source 454 may be disposed between the fluid collection assembly 400 and the fluid storage container 452.

The fluid storage container 452 is sized and shaped to retain bodily fluids therein. The fluid storage container 452 may include a bag (e.g., drainage bag), a bottle or cup (e.g., collection jar), or any other enclosed container for storing bodily fluids such as urine. In some examples, the conduit 414 may extend from the fluid collection assembly 400 and attach to the fluid storage container 452 at a first point therein. An additional conduit 414 may attach to the fluid storage container 452 at a second point thereon and may extend and attach to the vacuum source 454. Accordingly, a vacuum (e.g., suction) may be drawn through fluid collection assembly 400 via the fluid storage container 452. Bodily fluids, such as urine, may be drained from the fluid collection assembly 400 using the vacuum source 454.

The vacuum source 454 may include one or more of a manual vacuum pump, and electric vacuum pump, a diaphragm pump, a centrifugal pump, a displacement pump, a magnetically driven pump, a peristaltic pump, a wall mounted vacuum line, or any pump configured to produce a vacuum. The vacuum source 454 may provide a vacuum or suction to remove bodily fluids from the fluid collection assembly 400. In some examples, the vacuum source 454 may be powered by one or more of a power cord (e.g., connected to a power socket), one or more batteries, or even manual power (e.g., a hand operated vacuum pump). The vacuum source 454 may be a portable vacuum source. In some examples, the vacuum source 454 may be sized and shaped to fit outside of, on, or within the fluid collection assembly 400. For example, the vacuum source 454 may include one or more miniaturized pumps or one or more micro pumps. The vacuum sources 454 disclosed herein may include one or more of a switch, a button, a plug, a remote, or any other device suitable to activate the vacuum source 454.

In an embodiment, a method of collecting bodily fluid using a fluid collection assembly includes forming the fluid collection assembly into a selected shape by bending the shape memory material in the multiple lumen conduit thereof, positioning the opening of the fluid collection assembly adjacent to or on the urethra of a wearer, and receiving bodily fluid of the wearer in the fluid collection assembly (e.g., device).

The fluid collection assembly may include any of the fluid collection assemblies disclosed herein. Forming the fluid collection assembly into a selected shape by bending the shape memory material in the multiple lumen conduit thereof may include bending the fluid collection assembly into a generally arcuate shape that conforms to one or more surfaces of the wearer's body near the urethra along the sagittal plane.

Positioning the opening of the fluid collection assembly adjacent to or on the urethra of a wearer may include positioning the opening of the fluid collection assembly on or over the labia of a wearer. Positioning the opening of the fluid collection assembly adjacent to or on the urethra of a wearer may include positioning the opening of the fluid collection assembly on or over a penis (e.g., micropenis, buried penis) of a wearer.

Receiving bodily fluid of the wearer in the fluid collection assembly may include receiving and collecting urine into the porous material of the fluid collection assembly through the opening. Such collecting may include transporting the bodily fluid through the porous material toward the inlet of the multiple lumen conduit.

The method may include removing the bodily fluid from the chamber of the fluid collection assembly. Removing the bodily fluid from the chamber of the fluid collection assembly may include applying a vacuum force in the chamber via the conduit (e.g., fluid lumen of the conduit) using a vacuum source. Removing the bodily fluid from the chamber of the fluid collection assembly may include storing the removed bodily fluid in a fluid storage container.

The method may include using of the techniques or methods disclosed herein.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners. curved edges instead of straight edges, one or more protrusions extending therefrom, is oblong. is the same as the disclosed shape, etc.

What is claimed is:

1. A fluid collection assembly, comprising:
a fluid impermeable layer defining:
a chamber therein;
at least one opening therethrough; and
a fluid outlet;
at least one porous material disposed in the chamber;
a conduit disposed in the at least one porous material, the conduit including a multiple lumen configuration having a fluid lumen and a shape memory lumen;
a shape memory material disposed in the shape memory lumen; and
a stop plug and end plug disposed in the shape memory lumen, wherein the stop plug and end plug are disposed at opposite ends of the shape memory material in the shape memory lumen to prevent longitudinal movement of the shape memory material within the shape memory lumen.

2. The fluid collection assembly of claim 1 wherein the at least one porous material defines a bore configured to receive the conduit.

3. The fluid collection assembly of claim 1 wherein the at least one porous material includes a polyurethane foam.

4. The fluid collection assembly of claim 1 wherein the at least one porous material includes a three dimensional mesh material.

5. The fluid collection assembly of claim 1 wherein the at least one porous material includes a porous body material and a porous membrane material disposed over the porous body material.

6. The fluid collection assembly of claim 5 wherein the porous body material includes a polymer foam and the porous membrane material includes gauze.

7. The fluid collection assembly of claim 5 wherein the porous body material includes at least one vertical nonwoven material.

8. The fluid collection assembly of claim 7 wherein the at least one vertical nonwoven material includes at least one of polyester, polypropylene, polyurethane, nylon, cellulose, cotton, or bamboo.

9. The fluid collection assembly of claim 1 wherein the shape memory lumen is smaller than the fluid lumen.

10. The fluid collection assembly of claim 1 wherein the shape memory lumen is disposed in a gravimetrically low point in the fluid lumen.

11. The fluid collection assembly of claim 1 wherein the shape memory material includes a metal or a shape memory polymer.

12. The fluid collection assembly of claim 11 wherein the metal includes a copper alloy or a nickel alloy.

13. The fluid collection assembly of claim 1 wherein the stop plug and the end plug are constructed of a polymer, an adhesive, or a metal.

14. The fluid collection assembly of claim 1 wherein the stop plug and the end plug have a shape that is complementary to a shape of an inner wall of the conduit defining the shape memory lumen.

15. The fluid collection assembly of claim 1 wherein:
the stop plug is positioned within the shape memory lumen at a point within a longitudinal length of the fluid impermeable layer; and
the end plug is disposed within the shape memory lumen a distal end region of the conduit.

16. The fluid collection assembly of claim 1 wherein a distance between the stop plug and the end plug is longer than a length of the shape memory material by at least 0.25 cm.

17. A fluid collection system, comprising:
a fluid collection assembly including:
a fluid impermeable layer defining:
a chamber therein;
at least one opening therethrough; and
a fluid outlet;
at least one porous material disposed in the chamber;
a conduit disposed in the at least one porous material, the conduit including a multiple lumen configuration having a fluid lumen and a shape memory lumen;
a shape memory material disposed in the shape memory lumen; and
a stop plug and end plug disposed in the shape memory lumen, wherein the stop plug and end plug are disposed at opposite ends of the shape memory material in the shape memory lumen to prevent longitudinal movement of the shape memory material within the shape memory lumen;
a fluid storage container fluidly connected to the fluid collection assembly via a first conduit; and
a vacuum source fluidly connected to the fluid storage container via a second conduit effective to provide suction from the vacuum source to the chamber of the fluid collection assembly.

18. The fluid collection system of claim 17 wherein the vacuum source includes a wall mounted vacuum line or a portable vacuum source.

19. A method of collecting bodily fluid using a fluid collection assembly, the method comprising:
forming the fluid collection assembly into a selected shape by bending a shape memory material therein, the fluid collection assembly including:
a fluid impermeable layer defining:
a chamber therein;
at least one opening therethrough; and
a fluid outlet;
at least one porous material disposed in the chamber;
a conduit disposed in the at least one porous material, the conduit including a multiple lumen configuration having a fluid lumen and a shape memory lumen;
the shape memory material disposed in the shape memory lumen; and
a stop plug and end plug disposed in the shape memory lumen, wherein the stop plug and end plug are disposed at opposite ends of the shape memory material in the shape memory lumen to prevent longitudinal movement of the shape memory material within the shape memory lumen;
positioning the opening of the fluid collection assembly adjacent to or on a urethra of a wearer; and
receiving bodily fluid of the wearer in the fluid collection assembly.

20. The method of claim 19, further comprising removing bodily fluid from the fluid collection assembly.
